(19) United States
(12) Patent Application Publication
(10) Pub. No.: US 2004/0077659 A1
(43) Pub. Date: Apr. 22, 2004

(54) Method for treating ppar gamma mediated diseases or conditions

(76) Inventor: William Roland Oliver Jr, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/468,065

(22) PCT Filed: Feb. 21, 2002
(86) PCT No.: PCT/US02/05362

(51) Int. Cl. A61K 31/496; A61K 31/454; A61K 31/426

(52) U.S. Cl. 514/254.02; 514/326; 514/365

(57) ABSTRACT

The invention provides a method for treating a PPAR gamma method disease, risk factor or condition which comprises the administration of a compound or combination of compounds exhibiting agonist activity at human PPAR gamma, delta and alpha.

### FIELD OF THE INVENTION

The present invention relates to the treatment of diseases, risk factors, or conditions associated with peroxisome proliferator activated receptor (“PPAR”) gamma.

### BACKGROUND OF THE INVENTION

Peroxisome Proliferator Activated Receptors (PPARs) are orphan receptors belonging to the steroid/retinoid receptor superfamily of ligand-activated transcription factors. See, for example, Willson, T. M. and Wahli, W., Curr. Opin. Chem. Biol., (1997), Vol. 1, pp 235-241. Three mammalian PPARs have been identified which are termed PPAR-alpha, PPAR-gamma, and PPAR-delta. PPARs regulate expression of target genes by binding to DNA response elements as heterodimers with the retinoid X receptor. These DNA response elements (PPRE) have been identified in the regulatory regions of a number of genes encoding proteins involved in lipid metabolism and energy balance. The biological role of the PPARs in the regulation of lipid metabolism and storage has been recently reviewed. See, for example, Spiegelman, B. M., Diabetes, (1998), Vol. 47, pp 507-514, Schoonjans, K., Martin, G., Staels, B., and Auwerx, J., Curr. Opin. Lipidol., (1997), Vol. 8, pp 159-166, and Brun, R. P., Kim, J. B., Hu, E., and Spiegelman, B. M., Curr. Opin. Lipidol., (1997), Vol. 8, pp 212-218.

Treatment of type 2 diabetes mellitus usually begins with a combination of diet and exercise, with progression to oral hypoglycaemics (e.g. sulfonylureas) and in more severe cases, insulin. In the last decade, a class of compounds known as thiazolidinedlones (e.g. U.S. Pat. Nos. 5,089,514, 4,342,771, 4,367,234, 4,340,605, 5,306,726) have emerged as effective antidiabetic agents that enhance the insulin sensitivity of target tissues (skeletal muscle, liver, adipose) in animal models of type 2 diabetes mellitus and also reduce lipid and insulin levels in these animal models. It has been reported that thlazolidinediones are potent and selective activators of PPAR gamma and bind directly to the PPAR gamma receptor (J. M. Lehmann et. al., i J. Biol. Chem. 12953-12956, 270 (1995)), providing evidence that PPAR gamma is a possible target for the therapeutic actions of the thiazolidinediones.

Activators of the nuclear receptor PPARγ, for example troglitazone, have been shown in the clinic to enhance insulin-action, reduce serum glucose and have small but significant effects on reducing serum triglyceride levels in patients with type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

Activators of the nuclear receptor PPARγ have also been associated with certain undesired effects including fluid retention, hemodilution, weight gain, edema, and cardiac hypertrophy. See, for example, T. M. Willson, et al., J. Med. Chem., Vol. 43 (4), pages 527-550 (Feb. 24, 2000), and B. M. Spiegelman, Perspectives in Diabetes, Vol. 47, pages 507-514 (April 1998). The association with fluid retention and edema is of particular concern since edema and hemodilution are clinically associated with an increased risk of congestive heart failure. See, for example, Pioglitazone, S. P. Gillies and J. C. Dunn, Drugs, Vol. 60 (2), pages 333-343 (2000) and Rosiglitazone: an agent from the thiazolidinedione class for the treatment of type 2 diabetes, A. Cheng-Lai and A. Levine, Heart Des., Vol. 2(4), pages 326-333 (2000).

Castillo et al (1999), The EMBO J., Vol. 18 (13), pages 3676-3687 (1999) reports that ligand activation of PPAR gamma induces adipogenesis and increases insulin sensitivity while activation of other PPAR isoforms (alpha and delta) induces little or no fat differentiation. Bastie et al, J Biol Chem., Vol. 274 (3), pages 21920-21925 (1999) reports that the PPAR delta activation by fatty acids induced transcription of genes encoding fatty acid transporter, adipocyte lipid—binding protein and PPAR gamma demonstrating PPAR gamma gene expression is under the control of PPAR delta activated by fatty acids and may confer responsiveness to PPAR gamma agonist treatment, for example by thiazolidinediones.

International patent publication WO 98/05331 (Paterniti et. al) states that PPAR delta (formally known as NUC1 or PPARbeta) is known to repress the activity of PPAR alpha and PPAR gamma. The publication then suggests that it may be useful to reduce or relieve this repression by delta in order to enhance the effects, such as triglyceride lowering, of alpha or gamma.

International patent publication WO 01/00603 discloses compounds useful as agonists of PPAR delta. The publication states that PPAR delta agonists have several desirable clinical effects.

### BRIEF DESCRIPTION OF THE INVENTION

Briefly, in one aspect, the present invention discloses a method for treating a PPAR gamma mediated disease, risk factor, or condition in a human patient, comprising administration of a compound or combination of compounds exhibiting agonist activity at human PPAR (“hPPAR”) gamma, alpha, and delta. Compounds exhibiting agonist activity at all three hPPAR subtypes can be referred to as “PPAR pan agonists”. By “compound or combination of compounds” is meant that this hPPAR gamma, alpha, and delta activity can occur in one compound or in two or more separate compounds. We have now found that administration of a compound or combination of compounds exhibiting agonist activity at all three hPPAR subtypes is beneficial in the treatment of conditions associated with diseases, risk factors, or conditions associated with hPPAR gamma and in alleviating the symptoms associated therewith. The method of the present invention reduces the undesired effects associated with hPPAR gamma agonists, when compared to the action of a hPPAR gamma agonist alone, without reducing the desired effects associated with hPPAR gamma agonists.

According to another aspect of the invention we provide the use of a compound or combination of compounds exhibiting agonist activity at all three hPPAR subtypes for the manufacture of a medicament for the treatment of diseases, risk factors or conditions associated with hPPAR gamma and the alleviation of symptoms associated thereof.

Applicants have found that PPAR pan agonism reduces undesired effects of PPAR gamma, such as edema and weight gain associated with hPPAR gamma agonism, while not inhibiting the desired effects, such as diabetic glycemic control and improved lipid profile. As used herein “edema and weight gain associated with hPPAR gamma agonism” means that the edema or weight gain seen with PPAR pan agonism is significantly less than that which would be expected for a hPPAR gamma agonist. For example, average weight gains of less than 5% in a human taking a therapeutically effective amount of a PPAR gamma or PPAR pan agonist would be less than expected.

### DETAILED DESCRIPTION OF THE INVENTION

Diseases, risk factors, and conditions mediated by PPAR gamma include type I diabetes, type 2 or non-insulin dependent diabetes, syndrome X, (including metabolic syndrome), insulin resistance, heart failure, dyslipidemia including diabetic dyslipidemia and mixed dyslipidemia, hyperlipidemia, hypercholesterolemia, hypertension and cardiovascular disease, including atherosclerosis, arteriosclerosis and hypertriglyceridemia, epithelial hyperproliferative diseases including eczema and psoriasis and conditions associated with the lung and gut, osteoporosis, acne, cancer, and eating disorders or conditions such as obesity, bulimia, and anorexia nervosa.

In particular, the method of this invention is useful in the treatment and prevention of type 2 diabetes (NIDDM) and mixed dyslipidemia.

As used herein, by “agonist”, or “activating compound”, or “activator”, “exhibiting agonist activity” or the like, is meant those compounds which have a pKi of at least 6.0 (preferably at least 7.0) to the relevant PPAR, for example hPPAR delta, in the binding assay described below, and which achieve at least 30% (preferably at least 50%) activation of the relevant PPAR relative to the appropriate indicated positive control in the transfection assay described below at concentrations of 10⁻⁵ M or less (preferably 10⁻⁶ M or less). As discussed above, hPPAR pan agonist activity may reside in a single compound or in a combination of two or more compounds.

Preferred compounds with hPPAR pan activity include:

2-{4-[({4-{[4-(4-methoxyphenyl)-1-piperazinyl]methyl}-2-[4-(trifluoromethyl)phenyl]-1,3-thiazol-5-yl}methyl)sulfanyl]phenoxy}2-methylpropanoic acid,

2-{4-[({4-{[4-(4-chlorophenyl)-1-piperazinyl]methyl}-2-[4-(trifluoromethyl)phenyl]-1,3-thiazol-5-yl}methyl)sulfanyl]-2-methylphenoxy}propanoic acid,

{2-ethyl-4-[({4-{[4-methoxyphenyl)-1-piperazinyl]methyl}-2-[4-(trifluoromethyl)phenyl]-1,3-thiazol-5-yl}methyl)sulfanyl]phenoxy}acetic acid,

2-{4-[({4-{[4-(4-isopropoxyphenyl)-1-piperazinyl]methyl}-2-[4-(trifluoromethyl)phenyl]-1,3-thiazol-5-yl}methyl)sulfanyl]-2-methylphenoxy}propanoic acid, and

2-{4-[({2-[2-fluoro-4-(trifluoromethyl)phenyl]-4-methyl-1,3-thiazol-5-yl}methyl)sulfanyl]-2-methylphenoxy}-2-methylpropanoic acid.

A particularly preferred compound with hPPAR pan agonist activity is 2-{4-[({4-{[4-(4-methoxyphenyl)-1-piperazinyl]methyl}-2-[4-(trifluoromethyl)phenyl]-1,3-thiazol-5-yl}methyl)sulfanyl]phenoxy}-2-methylpropanoic acid.

It will also be appreciated by those skilled in the art that the compounds or combination of compounds may also be utlised in the form of a pharmaceutically acceptable salt or solvate thereof. The physiologically acceptable salts include conventional salts formed from pharmaceutically acceptable Inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. Those skilled in the art of organic chemistry will appreciate that many organic compounds can form complexes with solvents in which they are reacted or from which they are precipitated or crystallised. These complexes are known as “solvents”. For example, a complex with water is known as a “hydrate”. Solvates are within the scope of the invention.

The compounds and their pharmaceutically acceptable derivatives are conveniently administered in the form of pharmaceutical compositions. Such compositions may conveniently be presented for use in conventional manner In a mixture with one or more physiologically acceptable carriers or excipients.

While it is possible that compounds may be therapeutically administered as the raw chemical, it is preferable to present the active ingredient as a pharmaceutical formulation. The carrier(s) must be “acceptable” in the sense of being compatible with the other ingredients of the formulation and not deleterious to the recipient thereof.

Accordingly, the present invention further provides for a pharmaceutical formulation comprising a compound or combination of compounds exhibiting agonist activity at all three hPPAR subtypes or pharmaceutically acceptable salts or solvates thereof together with one or more pharmaceutically acceptable carriers therefore and, optionally, other therapeutic and/or prophylactic ingredients.

The formulations include those suitable for oral, parental (including subcutaneous e.g. by injection or by depot tablet, intradermal, intrathecal, intramuscular e.g. by depot and intravenous), rectal and topical (including dermal, buccal and sublingual) administration although the most suitable route may depend upon for example the condition and disorder of the recipient. The formulations may conveniently be presented in unit dosage form and may be prepared by any of the methods well known in the art of pharmacy. All methods include the step of bringing into association the compounds (“active ingredient”) with the carrier which constitutes one or more accessory ingredients. In general the formulations are prepared by uniformly and intimately bringing into association the active ingredients with liquid carriers or finely divided solid carriers or both and then, if necessary, shaping the product into the desired formulation.

Formulations suitable for oral administration may be presented as discrete units such as capsules, cachets or tablets (e.g. chewable tablets in particular for paediatric administration) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredients may also be presented as a bolus, electuary or paste.

A tablet may be made by compression or moulding, optionally with one or more accessory ingredients. Compressed tablets may be prepared by compressing in a suitable machine the active ingredients in a free-flowing form such as a powder or granules, optionally mixed with a other conventional excipients such as binding agents, (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate) or wetting agents, such as sodium lauryl sulfate. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The tablets may be coated according to methods well-known in the art.

Alternatively, the compounds may be incorporated into oral liquid preparations such as aqueous or oily suspensions, solutions, emulsions, syrups or elixirs, for example. Moreover, formulations containing these compounds may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or acacia; non-aqueous vehicles (which may include edible oils) such as almond oil, fractionated coconut oil, oily esters, propylene glycol or ethyl alcohol; and preservatives such as methyl or propyl p-hydroxybenzoates or sorbic acid. Such preparations may also be formulated as suppositories, e.g., containing conventional suppository bases such as cocoa butter or other glycerides.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilised) condition requiring only the addition of a sterile liquid carrier, for example, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter, hard fat or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

The compounds may also be formulated as depot preparations. Such long acting formulations may be administered by implantation (for example subcutaneously or intramuscularly) or by intramuscular Injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In addition to the ingredients particularly mentioned above, the formulations may include other agents conventional in the art having regard to the type of formulation in question, for example those suitable for oral administration may include flavouring agents.

It will be appreciated by those skilled in the art that reference herein to treatment extends to prophylaxis as well as the treatment of established diseases or symptoms. Moreover, it will be appreciated that the amount of a compound of the invention required for use in treatment will vary with the nature of the condition being treated and the age and the condition of the patient and will be ultimately at the discretion of the attendant physician or veterinarian. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day. The formulations according to the invention may contain between 0.1-99% of the active ingredient, conveniently from 30-95% for tablets and capsules and 3-50% for liquid preparations.

The compound or combination of compounds exhibiting agonist activity at all three hPPAR subtypes for use in the instant invention may be used in combination with other therapeutic agents for example, statins and/or other lipid lowering drugs for example MTP inhibitors and LDLr upregulators. The compounds of the invention may also be used in combination with antidiabetic agents, e.g. metformin, sulfonylureas. The compounds may also be used in combination with antihypertensive agents such as angiotensin antagonists e.g. telmisartan, calcium channel antagonists e.g. lacidipine and ACE inhibitors e.g. enalapril. The invention thus provides in a further aspect the use of a combination comprising a compound of formula (I) with a further therapeutic agent in the treatment of a hPPAR gamma mediated disease.

When the compound or combination of compounds exhibiting agonist activity at all three hPPAR subtypes are used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same formulation it will be appreciated that the compounds must be stable and compatible with each other and the other components of the formulation and may be formulated for administration. When formulated separately they may be provided in any convenient formulation, conveniently in such a manner as are known for such compounds in the art. When of a compound or combination of compounds exhibiting agonist activity at all three hPPAR subtypes is used in combination with a second therapeutic agent active against the same hPPAR gamma mediated disease, the dose of each compound may differ from that when the compound is used alone. Appropriate doses will be readily appreciated by those skilled in the art.

The invention will now be illustrated by way of the following Examples which should not be construed as constituting a limitation thereto.

### EXAMPLES

The following compounds were prepared and tested for their activity at the three hPPAR receptors:

2-{4-[({4-{[4-(4-methoxyphenyl)-1-piperazinyl]methyl}-2-[4-(trifluoromethyl)phenyl]-1,3-thiazol-5-yl}methyl)sulfanyl]phenoxy}-2-methylpropanoic acid.

2-{4-[({4-{[4-(4-chlorophenyl)-1-piperazinyl]methyl}-2-[4-(trifluoromethyl)phenyl]-1,3-thiazol-5-yl}methyl)sulfanyl]-2-methylphenoxy}propanoic acid,

{2-ethyl-4-[({4-{[4-(4-methoxyphenyl)1-piperazinyl]methyl}-2-[4-(trifluoromethyl)phenyl]-1,3-thiazol-5-yl}methyl)sulfanyl]phenoxy}acetic acid,

2-{4-[({4-{[4-(4-isopropoxyphenyl)-1-piperazinyl]methyl}-2-[4-(trifluoromethyl)phenyl]-1,3-thiazol-5-yl}methyl)sulfanyl]-2-methylphenoxy}propanoic acid, and

2-4-[({2-[2-fluoro-4-(trifluoromethyl)phenyl]-4-methyl-1,3-thiazol-5-yl}methyl)sulfanyl]-2-methylphenoxy}-2-methylpropanoic acid. Each of these five compounds are hPPAR pan agonists.

### Ethyl 4-(bromomethyl)-2-[4-(trifluoromethyl)phenyl]-1,3-thiazole-5-carboxylate

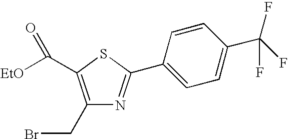

To a 2-L round-bottom flask equipped with an mechanical overhead stirrer, a reflux condenser and a N₂ inlet was added ethyl 4-methyl-2-[4-(trifluoromethyl)phenyl]-1,3-thiazole-5-carboxylate (85 g, 0.27 moles, 1.0 eq) and dry carbon tetrachloride (750 ml, 0.38M). Benzoyl peroxide (6.5 g, 10 mol %) was added at room temperature all at once as a solid. Freshly recrystallized N-bromo succinimide (52.72 g, 1.1 eq) was added as a solid and the reaction mixture was refluxed for 5 hrs. The reaction was monitored by ¹H NMR and was determined to be composed of a 9:1 mixture of mono-brominaton product (i.e. desired product) and di-bromination product with a 90% conversion. After cooling to 0° C. (to precipitate out the succinimide) the reaction was filtered through Celite and the solvent was removed under reduced pressure to yield a brown oil. The oil was crystallized using hexanes to yield 100 g (94%) of an off-white product of 90% purity.

¹H NMR (CDCl₃) 400 MHz δ 8.10(d, 2H, J=8.20 Hz), 7.72(d, 2H, J=8.20 Hz), 4.99(s, 2H), 4.40(q, 2H, J=7.18 Hz), 1.41(t, 3H, J=7.18 Hz), TLC(15% EtOAc/Hexanes) R_{f}=0.55

### Ethyl 4-(hydroxymethyl)-2-[4-(trifluoromethyl)phenyl]-1,3-thiazole-5-carboxylate

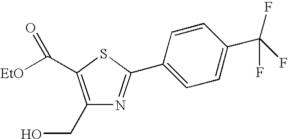

To a stirred solution of ethyl 4-(bromomethyl)-2-[4-(trifluoromethyl)phenyl]-1,3-thiazole-5-carboxylate (50 g, 0.127 moles, 1 eq) in dry DMF (300 ml) under a positive N₂ flow was added silver trifluoroacetate (42.02 g, 0.191 moles, 1.5 eq) all at once as a solid. This was stirred at room temperature for 3.5 hrs. The reaction was partitioned between ethyl ether (1.5 L) and water (500 ml). The phases were separated and the organic phase was washed twice with water (500 ml). After separation of the phases, the organic fraction was dried with Na₂SO₄, filtered and concentrated in vacuo. The crude trifluoroacetate product was used without characterization. Ethanol (300 ml) was added and the reaction was refluxed for 10 hrs. After cooling to room temperature the ethanol was removed in vacuo to yield 42 g (100%) of the title compound. The product was used without purification.

1H NMR (CDCl3) 400 MHz δ 8.09(d, 2H, J=8.20 Hz), 7.73(d, 2H, J=8.20 Hz), 5.09(s, 2H), 4.41(q, 2H, J=7.12 Hz), 1.40(t, 3H, J=7.12 Hz),

### Ethyl 4-[(tetrahydro-2H-pyran-2-yloxy)methyl]-2-[4-(trifluoromethyl)phenyl]-1,3-thiazole-5-carboxylate

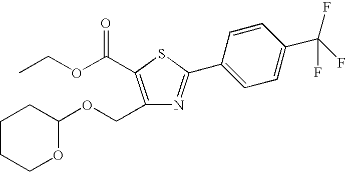

To a 1-L round-bottom flask equipped with a magnetic stir-bar and a N₂ inlet was added Ethyl 4-(hydroxymethyl)-2-[4-(trifluoromethyl)phenyl]-1,3-thiazole-5-carboxylate (42 g, 0.127 moles, 1 eq) and dry CH₂Cl₂ (300 ml) at room temperature. This was followed by the addition of 3,4-dihydro-2H-pyran (14 ml, 0.152 moles, 1.2 eq) as a neat liquid and pyridinium ρ-toluenesulfonate (6.4 g, 25.4 mmoles, 20 mol %). The reaction mixture was stirred at room temperature overnight (10 hrs). The volatiles were then removed in vacuo and the residue was purified by flash silica gel chromatography (10% EtOAc/Hexanes to 30% EtOAc/Hexanes) to yield 34 g (64%) of pure title compound.

¹H NMR (CDCl₃) 400 MHz δ 8.09(d, 2H, J=8.20 Hz), 7.69(d, 2H, J=8.20 Hz), 5.18(d, 1H, J=0.30 Hz), 4.99(d, 1H, J=0.30 Hz), 4.90(t, 1H, J=3.42 Hz), 4.36(q, 2H, J=7.12 Hz), 3.98(m, 1H), 3.56(m, 1H), 1.69(m, 6H), 1.37(t, 3H, J=7.12 Hz), TLC(30% EtOAc/Hexanes)=0.64

### 2-Fluoro-4-methylbenzenecarbethieamide

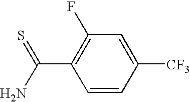

To a solution of 2-fluoro-4-(trifluoromethyl)benzonitrile (5.2 g, 27.5 mmol) in 50 mL methanol was added 10 ml of water (137.5 mmol) followed by NaSH.H₂O (7.7 g, 137.5 mmol). After heating at 50° C. for 12 hours, the solvent was removed in vacuo and the residue treated with water (200 ml) and extracted with EtOAc (2×150 mL). The organic layers were dried (MgSO₄) and the solvent evaporated to give crude residue which was purified on a Biotage FlashElute with a 40M silica cartridge, eluting with hexanes/ethyl acetate (4:1) to yield the title compound as a yellow solid (3.27 g, 53%).

MS m/z 224 (M+1); HPLC RT 2.013 (C18 4.6×60 mm, 1% MeOH/0-90% ACN/H₂O (0.1% TFA)/(50 mM TEA/TFA), 4 min @ 3 mL/min @ 254/220 nm).

### Ethyl 2-(2-fluoro-4-methylphenyl)-4-methyl-1,3-thiazole-5-carboxylate

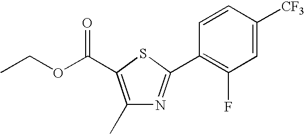

2-Fluoro-4-methylbenzenecarbothioamide was reacted with ethyl 2-chloro-3-oxobutanoate in refluxing ethanol overnight and evaporated. The residue was passed through a plug of silica gel with hexane:ethyl acetate (4:1) to afford the title compound as a light yellow solid after evaporation (71%).

MS m/z 333 (M+1)

### 4-[(Tetrahydro-2H-pyran-2-yloxy)methyl]-2-[4-(trifluoromethyl)phenyl]-1,3-thiazol-5-yl}methanol

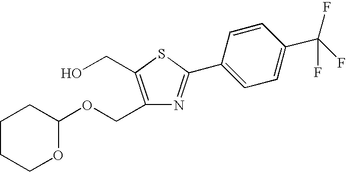

To a stirred solution of lithium aluminum hydride (95%, 3.3 g, 81.84 mmoles, 1 eq) in dry ethyl ether (300 ml) at 0° C. was added ethyl 2-(4-fluorophenyl)-4-[(tetrahydro-2H-pyran-2-yloxy)methyl]-1,3-thiazole-5-carboxylate) (34 g, 81.84 mmoles, 1 eq) in dry ethyl ether (50 ml) dropwise via an addition funnel maintaining the internal reaction temperature below 5° C. This was stirred at 0° C. for 1 hr. At 0° C. 3.5 ml water was added dropwise very carefully and was then allowed to warm to room temperature. This was followed by the addition 3.5 ml 5N NaOH and 10 ml water. The mixture was stirred at room temperature for 2 hrs. At this point a fine white precipitate formed. The reaction was filtered through Celite and the resulting aluminum salts were washed with 500 ml EtOAc. The ether/EtOAc solution was concentrated in vacuo to 30.6 g (100%) of titled alcohol.

¹H NMR (CDCl₃) 400 MHz δ 8.07(d, 2H, J=8.20 Hz), 7.72(d, 2H, J=8.20 Hz), 4.93(m, 4H), 4.78(t, 1H, J=3.32 Hz), 3.90(m, 1H), 3.61(m, 1H), 1.73(m, 6H), TLC(30% EtOAc/Hexanes)=0.20

### [2-(2-Fluoro-4-methylphenyl)-4-methyl-1,3-thiazol-5-yl]methanol

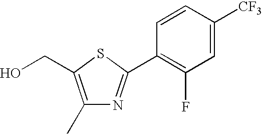

Ethyl 2-(2-fluoro-4-methylphenyl)4-methyl-1,3-thiazole-5-carboxylate was reacted as described in a the LiAlH₄ reduction procedure above to afford the title compound as a light yellow solid (83%)

MS m/z 291 (M+1)

### 5-(Chloromethyl)-4-[(tetrahydro-2H-pyran-2-yloxy)methyl]-2-[4-(trifluoromethyl)phenyl]-1,3-thiazole

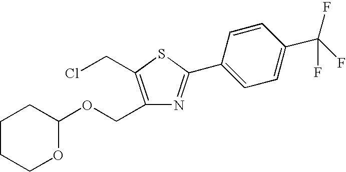

To a 500-ml round-bottom flask equipped with a magnetic stir-bar, an addition funnel and a N₂ inlet was added 4-[(tetrahydro-2H-pyran-2-yloxy)methyl]-2-[4-(trifluoromethyl)phenyl]-1,3-thiazol-5-yl}methanol (15 g, 40.17 mmoles, 1 eq) and dry CH₂Cl₂ (150 ml, 0.27M). Methaneasulfonyl chloride (3.73 ml, 48.20 mmoles, 1.2 eq) was added neat all at once followed by the dropwise addition of triethylamine (8.44 ml, 60.26 mmoles, 1.5 eq) over 10 minutes. This solution was stirred at room temperature for 1 hr. The reaction was transferred to a separatory funnel and washed with water and brine. After the phases were separated the CH₂Cl₂ fraction was dried over Na₂SO₄ and the solvent was removed in vacuo. This yielded 15.74 g (100%) of a brown oil. The crude product was used as is and required no purification.

¹H NMR (CDCl₃) 300 MHz δ 8.08(d, 2H, J=8.20 Hz), 7.73(d, 2H, J=8.20 Hz), 5.00(m, 3H), 4.80(m, 2H), 3.97(m, 1H), 3.64(m, 1H), 1.77(m, 6H), TLC(25% EtOAc/Hexanes) R_{f}=0.64

### 5-(Chloromethyl)-2-(2-fluoro-4-methylphenyl)-4-methyl-1,3-thiazole

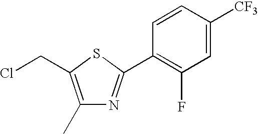

[2-(2-Fluoro-4-methylphenyl)-4-methyl-1,3-thiazol-5-yl]methanol was reacted with methanesulfonyl chloride as described above to afford the title compound as a light yellow solid (100%).

Rf of starting alcohol in 3:1 hexanes/ethyl acetate 0.25 Rf of chloride in 3:1 hexanes/ethyl acetate 0.75

### Ethyl (2-ethylphenoxy)acetate

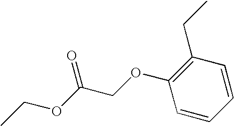

To a stirred solution of 2-ethylphenol (5 ml, 42.4 mmoles, 1 eq) in dry DMF (120 ml, 0.35M) was added potassium carbonate (6.45 g, 46.6 mmoles, 1.1 eq) and ethylbromoacetate (4.7 ml, 42.2 mmoles, 1 eq) and heated to 60° C. overnight. After cooling to room temperature the reaction mixture was partitioned between ethyl ether and 1N NaOH. The phases were separated and the organic portion was washed twice with 1N NaOH, twice with H₂O, brine, dried over Na₂SO₄, filtered and concentrated in vacuo to yield 7.2 g (82%) of product.

¹H NMR (CDCl₃) 400 MHz δ 7.14(m, 2H), 6.92(t,1H, J=8.24 Hz), 6.70(d, 1H, J=8.24 Hz), 4.62(s, 2H), 4.24(q, 2H, J=7.14 Hz), 2.70(q, 2H, J=7.51 Hz), 1.27(t, 3H, J=7.14 Hz), 1.21(t, 3H, J=7.51 Hz),

### Ethyl 2-[4-(chlorosulfonyl)phenoxy]-2-methylpropanoate

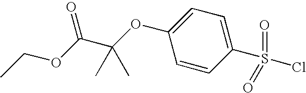

To a 3-L three-neck round-bottom flask equipped with a magnetic stir-bar, low temperature thermometer with thermometer adapter, addition funnel and a N₂ inlet was added ethyl 2-methyl-2-phenoxypropanoate (83 g, 0.399 moles, 1 eq) and dry CH₂Cl₂ (1 L, 0.4M). After cooling the reaction to 0° C. (ice bath) chlorosulfonic acid (26.5 ml, 0.399 moles, 1 eq) in dry CH₂Cl₂ (50 ml) was added dropwise over 30 minutes via addition funnel maintaining the internal temperature below 5° C. Following this dropwise addition the reaction was allowed to stir at 0° C. for 3 hours. The reaction was monitored by HPLC and after 3 hours complete conversion was observed [(C-18, 3 μm) 0%-95% Acetonitrile/Water over 8 minutes Rₜ=2.96 minutes]. At this point dry DMF (124 ml, 4 eq) was added slowly maintaining the internal temperature below 5° C. This was followed by the dropwise addition of thionyl chloride (43.77 ml, 0.599 moles, 1.5 eq) in dry CH₂Cl₂ (50 ml) over 25 minutes maintaining the internal temperature below 5° C. After stirring at 0° C. for 1.5 hours and monitoring by HPLC [(C-18, 3 μM) 0%-95% Acetonitrile/Water over 8 minutes Rₜ=5.97 minutes] the reaction was allowed to warm to room temperature. The reaction mixture was then washed with 0.1N HCl and the phases were separated, with discarding the aqueous fraction. The organic fraction was washed with 0.1N HCl, H₂O, brine and dried over Na₂SO₄. The solution was filtered and concentrated in vacuo to yield 119.95 g (98%) of pure sulfonyl chloride.

¹H NMR (CDCl₃) 400 MHz δ 7.89(d, 2H, J=9.31 Hz), 6.89(d, 2H, J=9.31 Hz), 4.21(q, 2H, J=7.16 Hz), 1.66(s, 6H), 1.20(t, 3H, J=7.16 Hz), HPLC (C-18, 3 μm) 0%-95% Acetonitrile/Water over 8 minutes Rₜ=5.97 minutes

### Ethyl 2-[4-(chlorosulfonyl)-2-methylphenoxy]-2-methylpropanoate

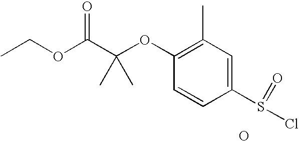

Ethyl 2-methyl-2-(2-methylphenoxy)propanoate was chlorosulfonated as described above.

¹H NMR (CDCl₃) 400 MHz δ 7.43(s, 1H), 7.34(d, 1H, J=8.28 Hz), 6.55(d, 1H, J=8.55 Hz), 4.18(q, 2H, J=7.08 Hz), 2.17(s, 3H), 1.54(s, 6H), 1.18(t, 3H, J=7.04 Hz)

### Ethyl [4-(chlorosulfonyl)-2-ethylphenoxy]acetate

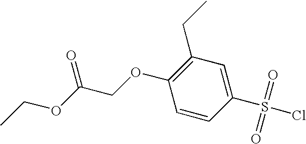

To a 250 ml round-bottom flask containing chlorosulfonic acid (30 ml) cooled to 0° C. was added ethyl (2-ethylphenoxy)acetate (7.2 g, 34.6 mmoles) dropwise. Once the addition was complete the ice-bath was removed and the reaction was allowed to warm to room temperature at which the reaction was stirred for 3 hours. The reaction was then slowly added to ice and, once the excess chlorosulfonic acid was quenched, the mixture was diluted with CH₂Cl₂ (200 ml). The phases were separated and the aqueous fraction was washed with CH₂Cl₂ twice. The combined organic fractions were dried over Na₂SO₄ and filtered and concentrated in vacuo to yield 7.2 g (70%) of crude product. The crude product was used with no purification.

¹H NMR (CDCl₃) 400 MHz δ 7.84(m, 2H), 6.79(d, 1H, J=8.24 Hz), 4.75(s, 2H), 4.26(q, 2H, J=7.14 Hz), 2.77(q, 2H, J=7.51 Hz), 1.26(m, 6H),

### Ethyl 2-[4-(chlorosulfonyl)-2-methylphenoxy]propanoate

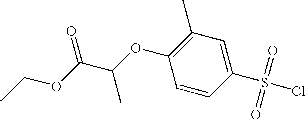

Ethyl 2-(2-methylphenoxy)propanoate was chlorosulfonated as described above.

¹H NMR (d6-DMSO) 300 MHz δ 7.44(m, 1H), 7.39(dd, 1H, J=8.23, 2.39 Hz), 6.74(d, 1H, J=8.23 Hz), 4.96(q, 1H, J=6.81 Hz), 4.13(q, 2H, J=7.08 Hz), 2.20(s, 3H), 1.54(d, 3H, J=6.81 Hz), 1.18(t, 3H, J=7.08 Hz),

### Ethyl 2-methyl-2-(4-sulfanylphenoxy)propanoate

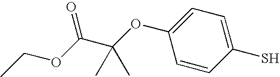

To a 3-L three-neck round-bottom flask equipped with an overhead mechanical stirrer, addition funnel and a N₂ inlet was added ethyl 2-[4-(chlorosulfonyl)phenoxy]-2-methylpropanoate (53 g, 0.173 moles, 1 eq) and absolute EtOH (500 ml). Tin powder (325 mesh, 123.06 g, 1.04 moles, 6 eq) was added as a solid. The overhead stirrer was adjusted so that the rotor is as close as possible to the bottom of the round-bottom flask and stirring speed was accelerated to a very high setting before adding the HCl to prevent the clumping of the tin metal. Hydrogen chloride (4N in dioxane, 300 ml) was added dropwise over the course of 1 hour. The reaction mixture was refluxed for 4 hours at which point the hot ethanolic solution was poured into a 2-L Erlenmeyer flask containing CH₂Cl₂ (1 L) and ice. After stirring for 10 minutes the biphasic mixture was filtered through Celite. After transferring to a separatory funnel the phases were separated and the aqueous fraction was washed with CH₂Cl₂ (2×100 ml). The combined organic fractions were dried over Na₂SO₄, filtered and concentrated in vacuo. A bright yellow oil with a white precipitate suspended resulted. This yellow mixture was dissolved in a minimum amount of CH₂Cl₂ and filtered once again through Celite to yield 30 g (75%) of a bright yellow oil.

¹H NMR (CD₃OD) 300 MHz δ 7.18(m, 2H), 6.73(d, 2H, J=8.00 Hz), 4.23(q, 2H, J=7.17 Hz), 3.69(s, 1H), 1.59(s, 6H), 1.26(t, 3H, J=7.17 Hz),

The following compounds were made in the same way and used without further purification.

### Ethyl 2-methyl-2-(2-methyl-4-sulfanylphenoxy)propanoate

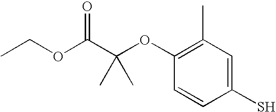

### Ethyl 2-(2-methyl-4-sulfanylphenoxy)propanoate

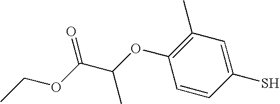

¹H NMR (CDCl₃) 400 MHz δ 7.12(d, 1H, J=2.39 Hz), 7.04(dd, 1H, J=8.37, 2.39 Hz), 6.56(d, 1H, J=8.37 Hz), 4.67(q, 1H, J=6.72 Hz), 4.19(q, 2H, J=7.12 Hz), 3.31(s, 1H), 2.22(s, 3H), 1.61(d, 3H, J=6.72 Hz), 1.23(t, 3H, J=7.12 Hz), TLC(20% EtOAc/Hexanes) R_{f}=0.60

### Ethyl (2-ethyl-4-sulfanylphenoxy)acetate

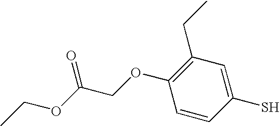

¹H NMR (CDCl₃) 400 MHz 7.13(d, 1H, J=2.20 Hz), 7.08(dd,1H, J=8.42, 2.38 Hz), 6.58(d, 1H, J=8.42 Hz), 4.59(s, 2H), 4.24(q, 2H, J=7.14 Hz), 3.33(s, 1H), 2.64(q, 2H, J=7.51 Hz), 1.28(t, 3H, J=7.14 Hz), 1.18(t, 3H, J=7.51 Hz),

### Ethyl 2-methyl-2-{4-[({4-[(tetrahydro-2H-pyran-2-yloxy)methyl]-2-[4-(trifluoromethyl)phenyl]-1,3-thiazol-5-yl}methyl)sulfanyl]phenoxy}propanoate

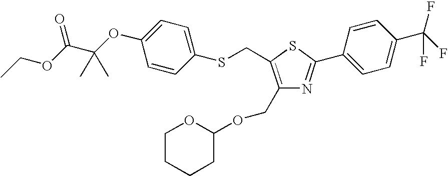

To a 250 ml round-bottom flask equipped with a magnetic stir-bar and N₂ inlet was added 5-(chloromethyl)-4-[(tetrahydro-2H-pyran-2-yloxy)methyl]-2-[4-(trifluoromethyl)phenyl]-1,3-thiazole (7.87 g, 20.09 mmoles, 1 eq) and dry CH₃CN (100 ml, 0.27M). Solid cesium carbonate (16.4 g, 50.22 mmoles, 2.5 eq) was added all at once followed by the quick addition of ethyl 2-methyl-2-(4-sulfanylphenoxy)propanoate (5.79 g, 24.11 mmoles, 1.2 eq) in dry CH₃CN (10 ml). The reaction was allowed to stir at room temperature for 2 hours at which point the solvent was removed under reduced pressure. The resulting residue was partitioned between EtOAc and 1N NaOH. After the phases were separated the organic fraction was washed with H₂O, brine and dried over Na₂SO₄. After filtration the volatiles were removed in vacuo to yield the titled compound in >100% yield. Sometimes because of the difficult separation between the thiophenol and the product, the crude product was carried forward without purification.

The following compounds were also made by alkylation of the corresponding thiophenol and 5-(chloromethyl)-4-[(tetrahydro-2H-pyran-2-yloxy)methyl]-2-[4-(trifluoromethyl)phenyl]-1,3-thiazole:

### Ethyl 2-{2-methyl-4-[({4-[(tetrahydro-2H-pyran-2-yloxy)methyl]-2-[4-(trifluoromethyl)phenyl]-1,3-thiazol-5-yl}methyl)sulfanyl]phenoxy}propanoate

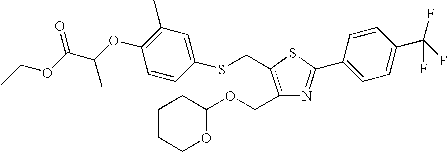

¹H NMR (CDCl₃) 300 MHz δ 8.04(d, 2H, J=8.23 Hz), 7.70(d, 2H, J=8.23 Hz), 7.27(d, 1H, J=2.39 Hz), 7.15(dd, 1H, J=8.49, 2.39 Hz), 6.60(d, 1H, J=8.49 Hz), 4.73(m, 3H), 4.51(d, 1H, J=0.21 Hz), 4.32(s, 2H), 4.20(q, 2H, J=7.17 Hz), 3.93(m, 1H), 3.60(m, 1H), 2.27(m, 3H), 1.71(m, 9H), 1.27(t, 3H, J=7.17 Hz), TLC(30% EtOAc/Hexanes)=0.73

### Ethyl {2-ethyl-4-[({4-[(tetrahydro-2H-pyran-2-yloxy)methyl]-2-[4-(trifluoromethyl)phenyl]-1,3-thiazol-5-yl}methyl)sulfanyl]phenoxy}acetate

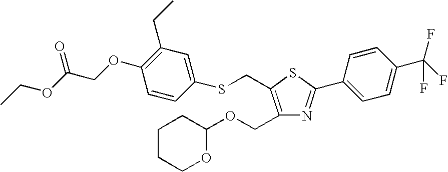

¹H NMR (CDCl₃) 400 MHz δ 7.98(d, 2H, J=8.24 Hz), 7.64(d, 2H, J=8.24 Hz), 7.20(d, 1H, J=2.20 Hz), 7.15(dd, 1H, J=8.42, 2.20 Hz), 6.60(d, 1H, J=8.42 Hz), 4.63(m, 4H), 4.42(d, 1H, J=0.27 Hz), 4.24(m, 4H), 3.87(m, 1H), 3.54(m, 1H), 2.64(q, 2H, J=7.51 Hz), 1.66(m, 6H), 1.26(t, 3H, J=7.14 Hz), 1.15(t, 3H, J=7.51 Hz),

### Ethyl 2-{4-[({2-[2-fluoro-4-(trifluoromethyl)phenyl]-4-methyl-1,3-thiazol-5-yl}methyl)sulfanyl]-2-methylphenoxy}-2-methylpropanoate

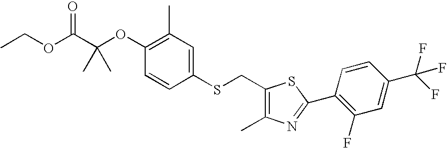

Ethyl 2-methyl-2-(2-methyl-4-sulfanylphenoxy)propanoate was alkylated with 5-(chloromethyl)-2-(2-fluoro-4-methylphenyl)-4-methyl-1,3-thiazole using a procedure analogous to that used above for the synthesis of ethyl 2-methyl-2-{4-[({4-[(tetrahydro-2H-pyran-2-yloxy)methyl]-2-[4-(trifluoromethyl)phenyl]-1,3-thiazol-5-yl}methyl)sulfanyl]phenoxy}propanoate.

MS(ES⁺) M+=527,

### Ethyl 2-{4-[({4-(hydroxymethyl)-2-[4-(trifluoromethyl)phenyl]-1,3-thiazol-5-yl}methyl)sulfanyl]phenoxy}-2-methylpropanoate

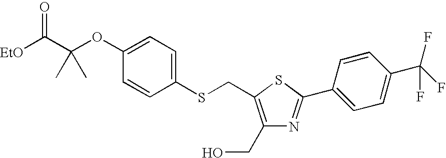

To a stirred solution of crude ethyl {2-methyl-4-[({4-[(tetrahydro-2H-pyran-2-yloxy)methyl]-2-[4-(trifluoromethyl)phenyl]-1,3-thiazol-5-yl}methyl)sulfanyl]phenoxy}acetate (11.98 g, 20.09 mmoles, 1 eq) in MeOH (100 ml, 0.20M) was added as a solid ρ-toluenesulfonic acid (800 mg, 25 mol %) at room temperature. The reaction mixture was stirred at room temperature for 3 hours. The MeOH was removed in vacuo and the residue was purified by silica gel chromatography (15% EtOAc/Hexanes to 30% EtOAc/Hexanes) to yield 8 g (78%) of pure titled alcohol.

¹H NMR (CDCl₃) 400 MHz δ 7.96(d, 2H, J=8.06 Hz), 7.65(d, 2H, J=8.06 Hz), 7.23(d, 2H, J=8.79 Hz), 6.73(d, 2H, J=8.79 Hz), 4.44(s, 2H), 4.17(m, 4H), 2.33(br s, 1H), 1.56(s, 6H), 1.21(t, 3H, J=7.14 Hz), TLC(30% EtOAc/Hexanes) R_{f}=0.32

### Ethyl {2-ethyl-4-[({4-(hydroxymethyl)-2-[4-(trifluoromethyl)phenyl]-1,3-thiazol-5-yl}methyl)sulfanyl]phenoxy}acetate

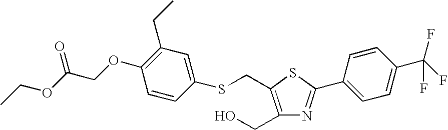

### Ethyl 2-{4-[({4-(hydroxymethyl)-2-[4-(trifluoromethyl)phenyl]-1,3-thiazol-5-yl}methyl)sulfanyl]-2-methylphenoxy}propanoate

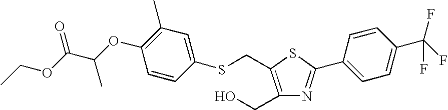

¹H NMR (CDCl₃) 300 MHz δ 8.00(d, 2H, J=8.23 Hz), 7.69(d, 2H, J=8.23 Hz), 7.22(d, 1H, J=2.39 Hz), 7.12(dd, 1H, J=8.23, 2.39 Hz), 6.59(d, 1H, J=8.23 Hz), 4.74(q, 1H, J=6.77 Hz), 4.51(s,2H), 4.19(m, 4H), 3.68(br s, 1H), 2.26(s, 3H), 1.65(d, 3H, J=6.77 Hz), 1.26(t, 3H, J=7.17 Hz), TLC(50% EtOAc/Hexanes) R_{f}=0.40

### Ethyl 2-[4-[({4-([4-(4-methoxyphenyl)-1-piperazinyl]methyl}-2-[4-(trifluoromethyl)phenyl]-1,3-thiazol-5-yl}methyl)sulfanyl]phenoxy}-2-methylpropanoate

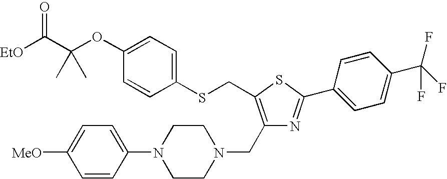

To a 500 ml 3-neck round-bottom flask equipped with a magnetic stir-bar, low temperature thermometer with thermometer adapter, addition funnel and N₂ inlet was added ethyl 2-{4-[({4-(hydroxymethyl)-2-[4-(trifluoromethyl)phenyl]-1,3-thiazol-5-yl}methyl)sulfanyl]phenoxy}-2-methylpropanoate (16 g, 31.28 mmoles, 1 eq) and dry CH₂Cl₂ (120 ml, 0.26M) and cooled to 0° C. Methanesulfonyl chloride (2.91 ml, 37.54 mmoles, 1.2 eq) was added neat all at once. Triethylamine (6.6 ml, 46.92 mmoles, 1.5 eq) was added dropwise over 20 minutes maintaining the internal temperature below 5° C. and was stirred at 0° C. for 30 minutes. The reaction mixture was transferred to a separatory funnel and washed with H₂O, brine and the organic fraction was dried over Na₂SO₄. After filtration the solvent was removed under reduced pressure to yield the corresponding mesylate in quantitative yield. Because of the unstable nature of the mesylate, the product was not characterized and was progressed onto the next stage without purification.

To the crude mesylate dissolved in dry THF (200 ml, 0.16M) was added 4-methoxyphenyl piperazine (13 g, 62.56 mmoles, 2 eq) and the reaction mixture was refluxed for 5 hours. After cooling to room temperature the solvent was removed in vacuo to yield a yellow solid residue. The residue was washed with a minimal amount of EtOAc and filtered through Celite to remove the 4-methoxyphenyl piperazine hydrochloride salt. The EtOAc was removed in vacuo and the resulting solid was filtered through a “plug” of silica gel using 30% EtOAc/Hexanes to yield 20.37 g (95%) of a light-yellow solid.

¹H NMR (CDCl₃) 400 MHz δ 7.96(d, 2H, J=8.24 Hz), 7.63(d, 2H, J=8.24 Hz), 7.27(d, 2H, J=8.79 Hz), 6.87(d, 2H, J=9.16 Hz), 6.80(d, 2H, J=9.16 Hz), 6.74(d, 2H, J=8.79 Hz), 4.32(s, 2H), 4.17(q, 2H, J=7.14 Hz), 3.73(s, 3H), 3.56(s, 2H), 3.06(br s, 4H), 2.59(br s, 4H), 1.55(s, 6H), 1.21(t, 3H, J=7.14 Hz), HPLC (C-18, 3 μm) 0%-95% Acetonitrile/Water over 8 minutes Rₜ=6.06 minutes

### Ethyl 2-{4-[({4-{[4-(4-chlorophenyl)-1-piperazinyl]methyl}-2-[4-(trifluoromethyl)phenyl]-1,3-thiazol-5-yl}methyl)sulfanyl]-2-methylphenoxy}propanoate

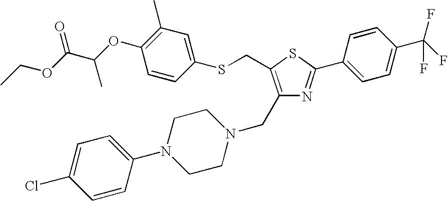

This compound was made using the same alkylation protocol as described above, from 4-chlorophenyl piperazine and ethyl 2-4-[({4-(hydroxymethyl)-2-[4-(trifluoromethyl)phenyl]-1,3-thiazol-5-yl}methyl)sulfanyl]-2-methylphenoxy}propanoate ¹H NMR (CDCl₃) 300 MHz δ 8.03(d, 2H, J=8.23 Hz), 7.70(d, 2H, J=8.23 Hz), 7.22(m, 4H), 6.86(d, 2H, J=9.03 Hz), 6.61(d, 1H, J=8.49 Hz), 4.73(q, 1H, J=6.81 Hz), 4.36(s, 2H), 4.18(q, 2H, J=7.08 Hz), 3.61(s, 2H), 3.17(m, 4H), 2.64(m, 4H), 2.27(s, 3H), 1.65(d, 3H, J=6.84 Hz), 1.27(t, 3H, J=7.08 Hz),

### Ethyl {2-ethyl-4-[({4-{[4-(4-methoxyphenyl)-1-piperazinyl]methyl}-2-[4-(trifluoromethyl)phenyl]-1,3-thiazol-5-yl}methyl)sulfanyl]phenoxy}acetate

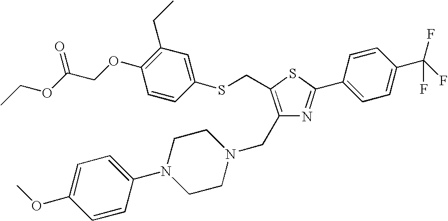

This compound was made using the same alkylation protocol as described above, from 4-methoxphenyl piperazine and ethyl {2-ethyl-4-[({4-(hydroxymethyl)-2-[4-(trifluoromethyl)phenyl]-1,3-thiazol-5-yl}methyl)sulfanyl]phenoxy}acetate.

¹H NMR (CDCl₃) 400 MHz δ 7.98(d, 2H, J=8.24 Hz), 7.65(d, 2H, J=8.24 Hz), 7.22(s, 1H), 7.17(d, 1H, J=8.42 Hz), 6.87(d, 2H, J=9.16 Hz), 6.81(d, 2H, J=9.16 Hz), 6.59(d, 1H, J=8.42 Hz), 4.60(s, 2H), 4.32(s, 2H), 4.22(q, 2H, J=7.14 Hz), 3.74(s, 3H), 3.53(s, 2H), 3.05(t, 4H, J=4.76 Hz), 2.62(m, 6H), 1.26(t, 3H, J=7.14 Hz), 1.16(t, 3H, J=7.33 Hz),

### Ethyl 2-{4-[({4-([(4-isopropoxyphenyl)-1-piperazinyl]methyl}-2-[4-(trifluoromethyl)phenyl]-1,3-thiazol-5-yl}methyl)sulfanyl]-2-methylphenoxy}propanoato

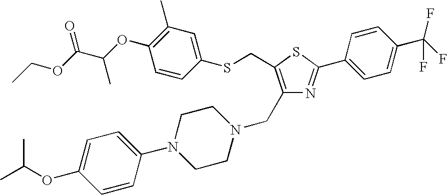

This compound was made using the same alkylation protocol as described above, from 4-isopropoxyphenyl piperazine and ethyl 2-{4-[({4-(hydroxymethyl)-2-[4-(trifluoromethyl)phenyl]-1,3-thiazol-5-yl}methyl)sulfanyl]-2-methylphenoxy}propanoate.

¹H NMR (CD₃OD) 400 MHz δ 7.96(d, 2H, J=8.28 Hz), 7.64(d, 2H, J=8.28 Hz), 7.18(d, 1H, J=2.24 Hz), 7.09(dd, 1H, J=8.45, 2.24 Hz), 6.81(d, 2H, J=9.14 Hz), 6.73(d, 2H, J=9.14 Hz), 6.57(d, 1H, J=8.45 Hz), 4.71(q, 1H, J=6.78 Hz), 4.36(m, 1H), 4.24(s, 2H), 4.06(q, 2H, J=7.16 Hz), 3.39(s, 2H), 2.92(t, 4H, J=4.57 Hz), 2.47(t, 4H, J=4.57 Hz), 2.11(s, 3H), 1.48(d, 3H, J=6.78 Hz), 1.19(d, 6H, J=6.21 Hz), 1.11(t, 3H, J=7.16 Hz),

### 2-{4-[({4-{[4-(4-Methoxyphenyl)-1-piperazinyl]methyl}-2-[4-(trifluoromethyl)phenyl]-1,3-thiazol-5-yl}methyl)sulfanyl]phenoxy}-2-methylpropanoic acid

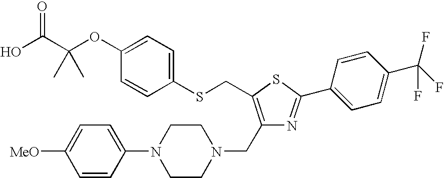

To a stirred solution of ethyl 2-{4-[({4-{[4-(4-methoxyphenyl)1-piperazinyl]methyl}-2-[4-(trifluoromethyl)phenyl]-1,3-thiazol-5-yl}methyl)sulfanyl]phenoxy}-2-methylpropanoate (77.0 g, 0.112 moles, 1 eq) in THF (600 ml, 0.19M) was added MeOH (50 ml) and a 1N LiOH solution (6.18 g in 250 ml H₂O, 2.3 eq). The mixture was refluxed for 5 hrs after which the THF was removed in vacuo. The residue was diluted with EtOAc and to it was added 1N HCl until a pH of about 5 was reached. The phases were separated and the organic fraction was concentrated in vacuo, then titrated with isopropyl acetate twice which was subsequently removed in vacuo each time. The crude product was then recrystallized from EtOH to yield 52 g (71%) of a white solid.

¹H NMR (CD₃OD) 400 MHz δ 8.08(d, 2H, J=8.24 Hz), 7.75(d, 2H, J=8.24 Hz,) 7.25(d, 2H, J=8.61 Hz), 6.94(d, 2H, J=9.16 Hz), 6.82(m, 4H), 4.28(s, 2H), 3.72(s, 3H), 3.59(s, 2H), 3.16(t, 4H, J=4.94 Hz), 2.96(t, 4H, J=4.94 Hz), 1.54(s, 6H), CHN Analysis: Theory (C, 60.26%; H, 5.21%; N, 6.39%) Found (C, 60.11%; H, 5.31%; N, 6.23%), HPLC (C-18, 3 μm) 0%-95% Acetonitrile/Water over 8 minutes Rₜ=5.48 minutes

The following compounds were also prepared from their corresponding esters following the procedure above.

### 2-{4-[({4-{[4-4-Chlorophenyl)-1-piperazinyl]methyl}-2-[4-(trlfluoromethyl)phenyl]-1,3-thiazol-5-yl}methyl)sulfanyl]-2-methylphenoxy}propanoic acid

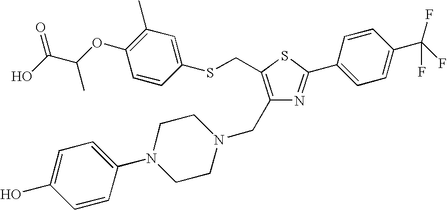

¹H NMR (CDCl₃) 400 MHz δ 10.42(s, 1H), 7.92(d, 2H, J=8.20 Hz), 7.64(d, 2H, J=8.20 Hz), 7.15(d, 2H, J=9.06 Hz), 7.01(d, 1H, J=2.20 Hz), 6.96(d, 1H, J=8.37 Hz), 6.72(d, 2H, J=9.06 Hz), 6.59(d, 1H, J=8.37 Hz), 4.64(q, 1H, J=6.78 Hz), 4.09(s, 2H), 3.58(d, 1H, J. 18 Hz), 3.49(d, 1H, J=0.18 Hz), 3.26(m, 4H), 3.05(m, 4H), 2.13(s, 3H), 1.56(d, 3H, J=6.78 Hz), MS(ES⁺) M+H=662.0, HPLC(C-18 3 μm) 1% MeOH/0-99% Acetonitrile/Water (0.1% TFA) 5 min run Rₜ=4.13

### {2-Ethyl-4-[({4-{[4-4-methoxyphenyl)-1-plperazinyl]methyl}-2-[4-trifluoromethyl)phenyl]-1,3-thiazol-5-yl}methyl)sulfanyl]phenoxy}acetic acid

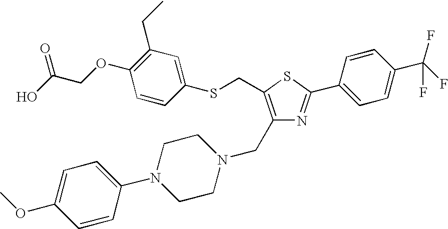

¹H NMR (CDCl₃) 400 MHz δ 7.97(d, 2H, J=8.28 Hz), 7.68(d, 2H, J=8.28 Hz), 7.15(dd, 1H, J=8.45, 2.24 Hz), 6.94(d, 1H, J=2.24 Hz), 6.88(d, 2H, J=9.14 Hz), 6.79(d, 2H, J=9.14 Hz), 6.72(d, 1H, J=8.45 Hz), 4.66(s, 2H), 4.08(s, 2H), 3.72(s, 3H), 3.32(m, 6H), 3.09(br s, 4H), 2.56(q, 2H, J=7.50 Hz, 1.08(t, 3H, J=7.50 Hz), MS(ES⁻) M−H=656.2

### 2-{4-[({4-{[4-(4-isopropoxyphenyl)-1-piperazinyl]methyl}-2-[4-(trifluoromethyl)phenyl]-1,3-thiazol-5-yl}methyl)sulfanyl]-2-methylphenoxy)propanoic acid

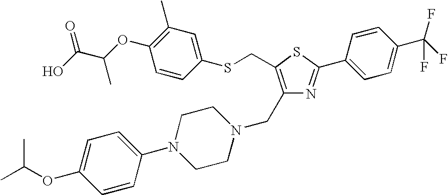

¹H NMR (CD₃OD) 400 MHz δ 8.13(d, 2H, J=8.06 Hz), 7.79(d, 2H, J=8.06 Hz), 7.13(m, 2H), 6.92(d, 2H, J=8.97 Hz), 6.81(d, 2H, J=8.97 Hz), 6.67(d, 1H, J=8.42 Hz), 4.61(q, 1H, J=6.78 Hz), 4.46(m,1H), 4.25(s, 2H), 3.56(s, 2H), 3.19(br s, 4H), 3.06(br s, 4H), 2.17(s, 3H), 1.55(d, 3H, J=6.78 Hz), 1.24(d, 6H, J=6.87 Hz), MS(ES₋) M−H=685.0

### 2-{4-[({2-[2-fluoro-4-(trifluoromethyl)phenyl]-4-methyl-1,3-thiazol-5-yl}methyl)sulfanyl]-2-methylphenoxy-}-2-methylpropanoic acid

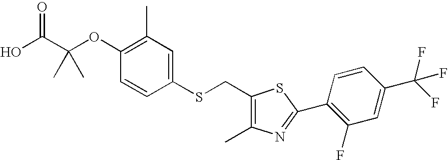

Ethyl 2-{4-[({2-[2-fluoro-4-(trifluoromethyl)phenyl]-4-methyl-1,3-thiazol-5-yl}methyl) sulfanyl]-2-methylphenoxy}-2-methylpropanoate was hydrolyzed using the general procedure as described above for 2-{4-[({4-{[4-(4-Methoxyphenyl)-1-piperazinyl]methyl}-2-[4-(trifluoromethyl)phenyl]-1,3-thiazol-5-yl}methyl)sulfanyl]phenoxy}-2-methylpropanoic acid, to afford the title compound as a cream solid (0.05 g, 17%).

¹H NMR (CD₃OD): δ 8.38 (t, 1 H), 7.65 (m, 2 H), 7.20 (s, 1 H), 7.12 (d, 1 H), 6.72 (d, 1 H), 4.24 (s, 2 H), 2.20 (s, 3 H), 2.17 (s, 3 H), 1.59 (s, 6 H); MS m/z 500 (M+1).

### Binding Assay

Compounds were tested for their ability to bind to hPPAR gamma, hPPAR alpha, or hPPAR delta using a Scintillation Proximity Assay (SPA). The PPAR ligand-binding domain (LBD) was expressed in E. coli as polyHis tagged fusion proteins and purified. The LBD was then labelled with biotin and immobilised on streptavidin-modified scintillation proximity beads. The beads were then incubated with a constant amount of the appropriate radioligand and variable concentrations of test compound, and after equilibration the radioactivity bound to the beads was measured by a scintillation counter. The radioligands used were: 3H-rosiglitazone for PPARgamma (Lehmann, J. M.; Moore, L. B.; Smith-Oliver, T. A.; Wilkison, W. O.; Willson, T. M.; Kliewer, S. A. J. Biol. Chem. 1995, 270, 12953-6.); radiolabelled 2-(4-(2-(2,3-Ditritio-1-heptyl-3-(2,4-difluorophenyl)ureido)ethyl)phenoxy)-2-methylbutanoic acid for hPPAR alpha (see (see Kliewer, S. A; Sundseth, S. S.; Jones, S. A.; Brown, P. J.; Wisely, G. B.; Koble, C.; Devchand, P.; Wahli, W.; Willson, T. M.; Lenhard, J. M.; Lehmann, J. M. Proc. Natl. Acad. Sci. U.S.A. 1997, 94, 4318-4323 and WO 00/08002) and labelled GW 2433 (see Brown, P. J et al. Chem. Biol. 1997, 4, 909-918, for the structure and synthesis of this ligand) for PPAR delta. The amount of nonspecific binding, as assessed by control wells containing 50 μM of the corresponding unlabeled ligand, was subtracted from each data point. For each compound tested, plots of ligand concentration vs. CPM of radioligand bound were constructed and apparent KI values were estimated from nonlinear least squares fit of the data assuming simple competitive binding. The details of this assay have been reported elsewhere (see, Blanchard, S. G. et. al. Development of a Scintillation Proximity Assay for Peroxisome Proliferator-Activated Receptor gamma Ligand Binding Domain. Anal. Biochem. 1998, 257, 112-119).

### Transfection assay

Compounds were screened for functional potency in transient transfection assays in CV-1 cells for their ability to activate the PPAR subtypes. A previously established chimeric receptor system was utilised to allow comparison of the relative transcriptional activity of the receptor subtypes on the same target gene and to prevent endogenous receptor activation from complicating the interpretation of results. See, for example, Lehmann, J. M.; Moore, L. B.; Smith-Oliver, T. A.; Wilkison, W. O.; Willson, T. M.; Kliewer, S. A., An antidiabetic thiazolidinedione is a high affinity ligand for peroxisome proliferator-activated receptor gamma (PPAR gamma), J. Biol. Chem., 1995, 270, 12953-6. The ligand binding domains for murine and human PPAR alpha, PPAR gamma, and PPAR delta were each fused to the yeast transcription factor GAL4 DNA binding domain. CV-1 cells were transiently transfected with expression vectors for the respective PPAR chimera along with a reporter construct containing five copies of the GAL4 DNA binding site driving expression of secreted placental alkaline phosphatase (SPAP) and beta-galactosidase. After 16 h, the medium was exchanged to DME medium supplemented with 10% delipidated fetal calf serum and the test compound at the appropriate concentration. After an additional 24 h, cell extracts were prepared and assayed for alkaline phosphatase and beta-galactosidase activity. Alkaline phosphatase activity was corrected for transfection efficiency using the beta galactosidase activity as an internal standard (see, for example, Kliewer, S. A., et. al. Cell 83, 813-819 (1995)). Rosiglitazone (BRL 49653) was used as a positive control in the hPPAR gamma assay. The positive control for PPAR delta assays was 2-{2-methyl-4-[({4-methyl-2-trifluoramethyl)phenyl]-1,3-thiazol-5-yl}methyl)sulfanyl]phenoxy}acetic acid (see WO 01/00603). The positive control in the hPPAR alpha assays was 2-[4-(2-(3-(4-fluorophenyl)-1-heptylureido)ethyl)-phenoxy]-2-methylpropionic acid, which can be prepared as described in Brown, Peter J., et. al. Synthesis Issue 7, 778-782 (1997), or patent publication WO 9736579.

### Diabetic Rat Studies

7-Day Study

The five PPAR pan agonist molecules prepared above were administered by oral gavage to genetically altered rodents that simulate the human disease of type 2 Diabetes Mellitus (Zucker Diabetic Fatty rats (ZDF fa/fa)). As a control a PPAR gamma agonist 2-[2-(methoxycarbonyl)anilino]-3-{4-[2-(5-methyl-2-phenyl-1,3-oxazol-4-yl)ethoxy]phenyl}propanoic acid was also tested. This PPAR gamma agonist can be prepared as described in Cobb, J. E., et al., N-(2-Benzoylphenyl)-L-tyrosine PPARg agonists. 3. Structure-activity relationship and optimization of the N-aryl substituent., J. Med. Chem. 1998, 41, 5055-5069. As an additional control, one group was treated with vehicle alone.

All five PPAR pan molecules effectively lowered glucose at doses of 30 mg/kg or less, resulting in decreases in glucose of 47% to 74% after 7 days of treatment, relative to same-age vehicle control animals. The PPAR gamma agonist at a dose of 6 mg/kg lowered glucose by 52% to 74% after 7 days of treatment, relative to same-age vehicle control animals.

The PPAR pan agonist molecules differed from the PPAR gamma agonist molecule in that there was little to no weight gain relative to control animals. Weight gain has been associated with edema in humans. In rodents, weight gain may be used as a potential surrogate marker for edema based on comparison of data generated with other insulin sensitising agents in rodents and their effects in humans. The PPAR gamma agonist increased body weight by 11% to 17% after 7 days of treatment, relative to same-age vehicle control animals. All five PPAR pan agonist molecules produced weight gain of less than 5% relative to same-age vehicle control animals after 7 days of treatment. Hemodilution, as measured by plasma hematocrit and total serum protein, was also much less with the five PPAR pan agonists than with the PPAR gamma agonist, following 7 days of treatment.

28-Day Study

While all five hPPAR pan agonist molecules were evaluated in the above study for 7 days, one of these pan agonists, 2-{4-[({4-{[4-(4-methoxyphenyl)-1-piperazinyl]methyl}-2-[4-(triflouromethyl)phenyl]-1,3-thiazol-5-yl}methyl)sulfanyl]phenoxy}-2-methylpropanoic acid, and the PPAR gamma agonist were tested for 28 days. Animals were treated earlier than in the 7-day study, before the development of diabetes as measured by increased plasma glucose. Measurements were obtained at 7 day intervals and the PPAR pan agonist molecule (♦) was compared to vehicle control (*) and the PPAR gamma agonist (□). The results are summarised in Charts 1, 2, and 3 shown below.

[image]

[image]

[image]

The data show that both the PPAR gamma agonist and the PPAR pan agonist lowered insulin and maintained glycemic control relative to vehicle treatment. The data also show that body weight increased with the PPAR gamma agonist by 120% from the start of dosing, whereas body weight gain with the PPAR pan agonist was not significantly different from the vehicle treated animals.

Hematocrit and total serum protein was also measured after 28 days. The group treated with vehicle had hematocrit (% RBC volume) of 49±0.3 and total serum protein (mg/dL) of 7.5±0.2. The group treated with the PPAR gamma agonist had hematocrit (% RBC volume) of 43±1.0 and total serum protein (mg/dL) of 6.0±0.1. The group treated with the PPAR pan agonist had hematocrit (% RBC volume) of 48±0.9 and total serum protein (mg/dL) of 7.2±0.2. These data show that hemodilution as indicated by hematocrit and total serum protein was evident at day 28 with the PPAR gamma agonist whereas the PPAR pan agonst was not significantly different from the vehicle group after 28 days.

### Primate Study

In addition to the above rat studies, a primate study was conducted using the PPAR pan agonist 2-{4-[({4-{[4-(4-methoxyphenyl)-1-piperazinyl]methyl}-2-[4-(trifluoromethyl)phenyl]-1,3-thiazol-5-yl}methyl)sulfanyl]phenoxy}-2-methylpropanoic acid. In that study, male and female Cynomolgus monkeys were dosed orally once daily for 63 or 64 days with doses projected to provide greater than 3× the serum concentration needed to lower insulin resistance and serum glucose in diabetic patients. The animals were dosed at 0 mg/kg (control), 1 mg/kg, 5 mg/kg, and 15 mg/kg of the PPAR pan agonist. The animals were carefully observed for signs of edema and or hemodilution. Methods of evaluation during the in-life portion of the study included visual observation of swelling, particularly around the eyes and or genitals by a Board Certified pathologist, as well as evaluation of clinical hematology (red blood cell volume) and clinical chemistries (total serum protein) as evidence of hemodilution. At necropsy, careful examination for gross evidence of edema was based on palpation and careful examination of all issue. Special emphasis was given to the gross evaluation of subcutaneous tissue, all fat stores, lungs and all serous cavities. No evidence of edema was present in any of the tissues examined histologically. There were no signs or suggestions of edema or hemodilution during the in-life or necropsy portions of the study.

What is claimed is:

1. A method for treating a hPPAR gamma mediated disease, risk factor, or condition in a human comprising the step of administering a therapeutically effective amount of a compound or combination of compounds exhibiting agonist activity at hPPAR gamma, alpha, and delta.

2. The method of claim 1 comprising administration of a compound that is a hPPAR pan agonist.

3. The method of claim 1 wherein said disease, risk factor, or condition is diabetes, metabolic syndrome, impaired glucose tolerance, syndrome X, mixed dyslipidemia, or glycemic control.

4. A method for achieving the glycemic control associated with hPPAR gamma agonists without the edema also associated with hPPAR gamma agonists comprising the step of administering a therapeutically effective amount of a compound or combination of compounds exhibiting agonist activity at hPPAR gamma, alpha, and delta.

5. A method for achieving the glycemic control associated with hPPAR gamma agonists without the weight gain also associated with hPPAR gamma agonists comprising the step of administering a therapeutically effective amount of a compound or combination of compounds exhibiting agonist activity at hPPAR gamma, alpha, and delta.

6. A method for achieving the glycemic control associated with hPPAR gamma agonists without the hemodilution also associated with hPPAR gamma agonists comprising the step of administering a therapeutically effective amount of a compound or combination of compounds exhibiting agonist activity at hPPAR gamma, alpha, and delta.

7. The method of claim 2 wherein said compound is selected from the group consisting of:
2-{4-[({4-{[4-(4-methoxyphenyl)-1-piperazinyl]methyl}-2-[4-(trifluoromethyl)phenyl]-1,3-thiazol-5-yl}methyl)sulfanyl]-2-methylphenoxy}propanoic acid,
2-{4-[({4-{[4-(4-chlorophenyl)-1-piperazinyl]methyl}-2-[4-(trifluoromethyl)phenyl]-1,3-thiazol-5-yl}methyl)sulfanyl]-2-methylphenoxy}propanoic acid,
{2-ethyl-4-[({4-{[4-(4-methoxyphenyl)-1-piperazinyl]methyl}-2-[4-(trifluoromethyl)phenyl]-1,3-thiazol-5-yl}methyl)sulfanyl]phenoxy}acetic acid,
2-{4-[({4-{[4-(4-isopropoxyphenyl)-1-piperazinyl]methyl}-2-[4-(trifluoromethyl)phenyl]-1,3-thiazol-5-yl}methyl)sulfanyl]-2-methylphenoxy}propanoic acid,
2-{4-[({2-[2-fluoro-4-(trifluoromethyl)phenyl]-4-methyl-1,3-thiazol-5-yl}methyl)sulfanyl]-2-methylphenoxy)-2-methylpropanoic acid, and
salts and solvates thereof.

8. The method of claim 2 wherein said compound is 2-{4-[({4-{[4-(4-methoxyphenyl)-1-piperazinyl]methyl}-2-[4-(trifluoromethyl)phenyl]-1,3-thiazol-5-yl}methyl)sulfanyl]phenoxy}-2-methylpropanoic acid or a salt or solvate thereof.

9. A method for identifying compounds that will be useful for the treatment of a PPAR-gamma mediated disease, risk factor, or condition in a human comprising the step of determining whether the compound exhibits agonist activity at all three hPPAR subtypes.

10. A method for treating a PPAR-gamma mediated disease, risk factor, or condition in a human comprising the step of administration of a therapeutically effective amount of a compound or compounds identified using the method of claim 9.
